(19) United States
(12) Patent Application Publication
(10) Pub. No.: US 2024/0254077 A1
(43) Pub. Date: Aug. 1, 2024

(54) METHODS OF TREATING CHRONIC OBSTRUCTIVE PULMONARY DISEASE INCLUDING COMPOUNDS USEFUL THEREIN

(71) Applicant: The Trustees of Columbia University in the City of New York, New York, NY (US)

(72) Inventor: Jeanine D'Armiento, New York, NY (US)

(73) Assignee: The Trustees of Columbia University in the City of New York, New York, NY (US)

(21) Appl. No.: 18/596,300

(22) Filed: Mar. 5, 2024

Related U.S. Application Data
(63) Continuation of application No. PCT/US2022/076025, filed on Sep. 7, 2022.
(60) Provisional application No. 63/241,509, filed on Sep. 7, 2021.

(51) Int. Cl. C07C 275/10; A61K 31/17; A61K 31/18; C07C 311/04; C07C 311/17

(52) U.S. Cl. CPC C07C 275/10; A61K 31/17; A61K 31/18; C07C 311/04; C07C 311/17

(57) ABSTRACT

The present invention relates to the treatment of chronic obstructive pulmonary disease (COPD). More specifically, embodiments of the invention provide a pharmaceutical carrier and a compound that inhibit the induction of MMP-1 expression by cigarette smoke.

This application is a continuation of PCT International Application No. PCT/US2022/076025, filed Sep. 7, 2022, claiming the benefit of U.S. Provisional Application No. 63/241,509, filed Sep. 7, 2021, the contents of which are hereby incorporated by reference into the subject application.

This invention was made with government support under TR001873, and HL086936 awarded by the National Institutes of Health. The government has certain rights in the invention.

Throughout this application, various publications are referred to by first author and year of publication. Full citations of these references can be found following the Examples. The disclosures of these publications are hereby incorporated by reference into this application in their entireties in order to more fully describe the state of the art to which this invention pertains.

### BACKGROUND OF THE INVENTION

Chronic obstructive pulmonary disease (COPD) is an enormous unmet medical need with no disease modifying treatments available. Present therapies provide symptomatic treatment, but no drug treats its cause or slows its progression. Lack of effective therapy results in a vicious feedback loop of chronic pulmonary decline punctuated by periods of acute disease exacerbation secondary to infection that accelerates loss of respiratory function. Ultimately, patients' ability to perform activities of daily living becomes compromised, leading to permanent disability. Death results from the respiratory failure/infection itself or other related comorbidities, such as heart failure.

The most common cause of COPD is cigarette smoking—a behavior whose prevalence in the U.S. has remained fairly constant but continues to rise worldwide. However a host of genetic and pollutant related causes also contribute. In the U.S. alone, each year this disease results in more than 100,000 deaths, is responsible for over 600,000 hospitalizations and over 15 million physician office visits, and causes approximately 150 million days of disability. Worldwide it is estimated that about 600 million adults have COPD, of which 24 million live in the U.S. Global mortality is estimated at 3.17 million, accounting for 5% of all deaths worldwide.

Present interventions used for COPD serve to ameliorate the symptoms of the disease but do not address its overall course. The physiologic hallmark of COPD is fixed airway obstruction with a progressive decline in the forced expiratory volume in one second (FEV₁). Bronchodilators, including anticholinergics (e.g., Atrovent®, Spiriva®) and R-adrenergic agonists (e.g., albuterol, Opened®), relax airway smooth muscle and appear to decrease dyspnea, increase FEV₁, and decrease the frequency of reported exacerbations in certain populations. The effect of bronchodilators is short-lived, however, and these agents do not slow the progression of the disease as measured by a long-term decline in FEV₁. The regular use of inhaled corticosteroids (e.g., Flovent®) reduces symptoms, frequency of exacerbations, and numbers of outpatient physician visits in patients with moderate or severe COPD, but does not affect the rate of decline in post-bronchodilator FEV₁. However, chronic use of systemic corticosteroids does not improve the course of COPD, and may increase mortality. While these approaches improve patient quality of life in the short term, they are still symptomatic in nature. There is no treatment yet available to address chronic pulmonary decline in the COPD patient and slow disease progression.

Smokers are ten times more likely than non-smokers to die of COPD. Smoking cessation is the only intervention of proven value in early-stage COPD, however, even with cessation, the destructive process initiated by cigarette smoking continues emphasizing the need for therapies targeted towards smoke induced inflammation and lung destruction.

The availability of a disease modifying therapy for COPD has the potential to alter the delivery, management and cost of care for multiple stakeholders in the COPD space. First and foremost, patients stand to benefit from decreased mortality and functional decline with a greatly improved quality of life. Re-education of family practitioners, internist and pulmonologists will be required to ensure prompt intervention with therapy in addition to typical symptomatic and exacerbation related treatment. With decreased rates of functional decline and exacerbation expected, general hospital and ICU related admission for care are expected to drop, reducing hospital costs and freeing resources for use elsewhere. Beds in rehab centers and home care services may also see a decline in patient volume, which should relieve strain on resources. However, some of these stakeholders may see this as a loss of business. Payors, specifically Medicare and Medicaid, which cover 51% and 25% of the cost of COPD treatment, respectively, also stand to see a significant decrease in payouts for COPD care, likely permitting them to shift resources to other diseases or assist with payment elsewhere.

There remains a need in the art for new therapies that more effectively treat subjects afflicted with lung diseases, such as COPD.

### SUMMARY OF THE INVENTION

The present invention provides a compound having the structure:

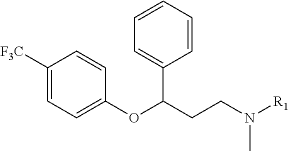

wherein
R₁ is —C(O)—NH-(alkyl) or —SO₂-(alkyl)
   wherein the alkyl may be straight, branched or cyclic,
or —SO₂-(aryl)
   wherein the aryl is optionally substituted with one or more -(alkyl), —O-(alkyl), halogen, —CF₃, —OCF₃ or —CN;
or a pharmaceutically acceptable salt thereof.

The present invention provides a method of treating a subject afflicted with a lung disease comprising administering to the subject an effective amount of a compound having the structure:

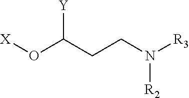

wherein
X is aryl or heteroaryl, which is optionally substituted with one or more -(alkyl), —O-(alkyl), halogen, —CF₃, —OCF₃ or —CN, wherein the alkyl may be straight, branched or cyclic;
Y is aryl or heteroaryl, which is optionally substituted with one or more -(alkyl), —O-(alkyl), halogen, —CF₃, —OCF₃ or —CN, wherein the alkyl may be straight, branched or cyclic;
R₂ is H, -(alkyl), -(alkenyl), -(alkynyl) or -cycloalkyl, wherein the alkyl, alkenyl or alkynyl may be straight, branched or cyclic; and
R₃ is H, -(alkyl), -(alkenyl), -(alkynyl), —C(O)—NH-(alkyl), —C(S)—NH-(alkyl), —C(O)-(alkyl), —SO₂-(alkyl) or —SO₂-(aryl), wherein the alkyl, alkenyl or alkynyl may be straight, branched or cyclic, and the aryl is optionally substituted with one or more -(alkyl), —O-(alkyl), halogen, —CF₃, —OCF₃ or —CN;
or a pharmaceutically acceptable salt thereof, so as to thereby treat the subject.

### BRIEF DESCRIPTION OF THE DRAWINGS

FIG. 1: Schematic of Screening Assay. Schematic of screening assay using cigarette smoke responsive MMP-1 promoter region.

FIG. 2: Fluoxetine inhibits MMP-1 production and TLR-4 signaling in CSE exposed SAEC cultures. (A) Fluoxetine treatment decreases MMP1 present in the media of 5% CSE exposed SAEC cultures. (B) As assessed by PCR, fluoxetine inhibits expression of MMP1 transcript in 5% CSE exposed SAEC cultures. Fluoxetine abrogates the CSE induced increase in TLR-4 expression (C) and activation of its downstream effector phospho-IRAK (D).

FIG. 3: Oral duloxetine treatment in smoke exposed rabbits reduces MMP-1 secretion and lung destruction. (A) Duloxetine treatment decreases MMP1 present in the BALF of smoke exposed rabbits. (B) Duloxetine treated rabbits display a significantly reduced mean linear intercept as compared to untreated smoke exposed animals. (C) Representative H&E stains for each treatment group.

FIG. 4: Oral fluoxetine treatment in smoke exposed mice reduces inflammatory BALF cell counts. (A) Fluoxetine treatment decreases the cell count in smoke exposed animals. (B) Representative cytospin DifQuik stains for each experimental group.

FIG. 5: Chemical structures of L48H37 (A), curcumin (B) and fluoxetine (C) showing similarity of aromatic ring placement. The red box indicates where fluoxetine has been modified to generate the initial library of derivatives.

FIG. 6: MMP1 activity—fluoexetine derivatives on SAEC. Comparison of Derivatives 1-6 on MMP1 concentration.

FIG. 7: Dose response curve analysis showing Fluoxetine derivative treatment reduces IL-8 expression in CSE treated SAEC. (A) Fluoxetine. (B) Derivative 2. (C) Derviative 5. SAEC cultures were treated with 5% CSE and varying doses of Fluoxetine, Derivative 2 and Derivative 5, respectively. Treatment with the derivatives decreased MMP1 induction after cigarette some treatment.

FIG. 8: Dose response curve analysis showing Fluoxetine derivative treatment reduces MMP1 expression in CSE treated SAEC. (A) Fluoxetine. (B) Derivative 2. (C) Derviative 5. SAEC cultures were treated with 5% CSE and varying doses of Fluoxetine, Derivative 2 and Derivative 5, respectively. Treatment with the derivatives decreased MMP1 induction after cigarette smoke treatment.

### DETAILED DESCRIPTION OF THE INVENTION

Each embodiment disclosed herein is contemplated as being applicable to each of the other disclosed embodiments. Thus, all combinations of the various elements described herein are within the scope of the invention.

### Embodiments of the Invention

The present invention provides compounds with improved potency, lower toxicity, and higher selectivity compared to fluoxetine.

The present invention provides compounds and methods for preventing the destruction of lung in a lung disease. In an embodiment, the lung disease is COPD. In an embodiment, the lung disease is emphysema. Additionally, compounds of the invention may be used prophylactically, to prevent the development of the lung disease.

The present invention provides a compound having the structure:

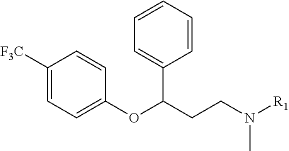

wherein
R₁ is —C(O)—NH-(alkyl), —SO₂-(alkyl),
   wherein the alkyl may be straight, branched or cyclic,
or —SO₂-(aryl)
   wherein the aryl is optionally substituted with one or more -(alkyl), —O-(alkyl), halogen, —CF₃, —OCF₃ or —CN;
or a pharmaceutically acceptable salt thereof.

In an embodiment, R₁ is —C(O)—NH—(C₁-C₈ alkyl) or —SO₂—(C₁-C₈ alkyl). The present invention also provides a compound having the structure:

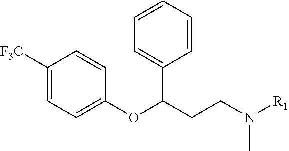

wherein
R₁ is —C(O)—NH-(alkyl) or —SO₂-(alkyl),
   wherein the alkyl may be straight, branched or cyclic
or a pharmaceutically acceptable salt thereof.

In an embodiment, R₁ is —C(O)—NH—(C₁-C₈ alkyl) or —SO₂—(C₁-C₈ alkyl).

The present invention also provides a compound having the structure:

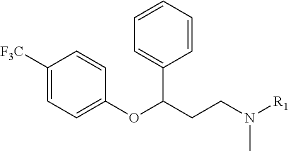

wherein
R₁ is —C(O)—NH-(ethyl) or —SO₂-(methyl);
or a pharmaceutically acceptable salt thereof.

In some embodiments, R₁ is —C(O)—NH-(ethyl).

In some embodiments, R₁ is —SO₂-(methyl).

In some embodiments, R₁ is —SO₂-(4-methylphenyl).

In some embodiments, the compound has the structure:

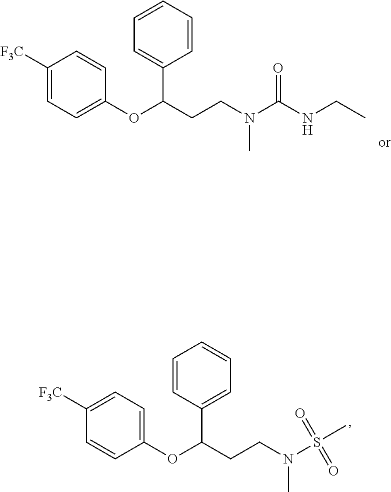 [SMILES: CCNC(=O)N(C)CCC(Oc1ccc(C(F)(F)F)cc1)c1ccccc1 ; CN(CCC(Oc1ccc(C(F)(F)F)cc1)c1ccccc1)S(C)(=O)=O]

or a pharmaceutically acceptable salt thereof.

In some embodiments, the compound has the structure:

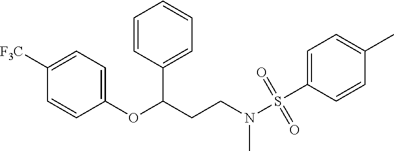 [SMILES: Cc1ccc(S(=O)(=O)N(C)CCC(Oc2ccc(C(F)(F)F)cc2)c2ccccc2)cc1]

or a pharmaceutically acceptable salt thereof.

The present invention also provides a pharmaceutical composition comprising a compound as described above and a pharmaceutically acceptable carrier.

The present invention also provides a method of treating a subject afflicted with a lung disease comprising administering to the subject an effective amount of a compound having the structure:

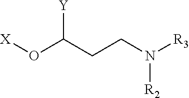

wherein
X is aryl or heteroaryl, which is optionally substituted with one or more -(alkyl), —O-(alkyl), halogen, —CF₃, —OCF₃ or —CN, wherein the alkyl may be straight, branched or cyclic;
Y is aryl or heteroaryl, which is optionally substituted with one or more -(alkyl), —O-(alkyl), halogen, —CF₃, —OCF₃ or —CN, wherein the alkyl may be straight, branched or cyclic;
R₂ is H, -(alkyl), -(alkenyl), -(alkynyl) or -cycloalkyl, wherein the alkyl, alkenyl or alkynyl may be straight, branched or cyclic; and
R₃ is H, -(alkyl), -(alkenyl), -(alkynyl), —C(O)—NH-(alkyl), —C(S)—NH-(alkyl), —C(O)-(alkyl), —SO₂-(alkyl) or —SO₂-(aryl), wherein the alkyl, alkenyl or alkynyl may be straight, branched or cyclic, and the aryl is optionally substituted with one or more -(alkyl), —O-(alkyl), halogen, —CF₃, —OCF₃ or —CN;
or a pharmaceutically acceptable salt thereof, so as to thereby treat the subject.

In some embodiments, X is aryl, optionally substituted with one or more -(alkyl), —O-(alkyl), halogen, —CF₃, —OCF₃ or —CN, wherein the alkyl may be straight, branched or cyclic.

In some embodiments, Y is aryl, optionally substituted with one or more -(alkyl), —O-(alkyl), halogen, —CF₃, —OCF₃ or —CN, wherein the alkyl may be straight, branched or cyclic.

In some embodiments,
X is phenyl, or
Y is phenyl, or
X and Y are phenyl.

In some embodiments, R₂ is -(alkyl).

In some embodiments, R₃ is -(alkyl), —C(O)—NH-(alkyl), —C(S)—NH-(alkyl), —C(O)-(alkyl), —SO₂-(alkyl) or —SO₂-(aryl).

In some embodiments, the compound has the structure:

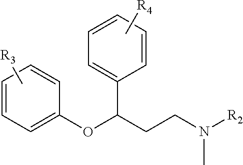

wherein
R₂ is -(alkyl), —C(O)—NH-(alkyl), —C(S)—NH-(alkyl), —C(O)-(alkyl), —SO₂-(alkyl) or —SO₂-(aryl); and
R₃ and R₄ are each independently H, -(alkyl), —O-(alkyl), halogen, —CF₃, —OCF₃ or —CN, wherein the alkyl may be straight, branched or cyclic,
or a pharmaceutically acceptable salt thereof.

In some embodiments, the compound has the structure:

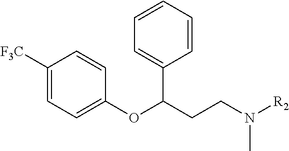

wherein
R₂ is -(alkyl), —C(O)—NH-(alkyl), —C(S)—NH-(alkyl), —C(O)-(alkyl), —SO₂-(alkyl) or —SO₂-(aryl),
or a pharmaceutically acceptable salt thereof.

In some embodiments, R₂ is -(n-butyl), —C(O)—NH-(ethyl), —C(S)—NH-(ethyl), —C(O)-(ethyl), —SO₂-(methyl) or —SO₂-(4-methylphenyl)

In some embodiments, the compound has the structure:

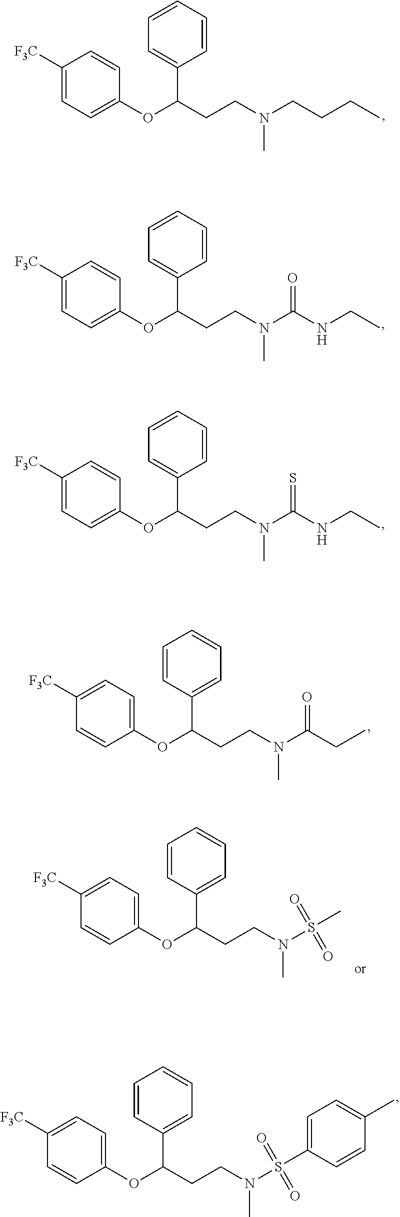 [SMILES: CCCCN(C)CCC(Oc1ccc(C(F)(F)F)cc1)c1ccccc1 ; CCNC(=O)N(C)CCC(Oc1ccc(C(F)(F)F)cc1)c1ccccc1 ; CCNC(=S)N(C)CCC(Oc1ccc(C(F)(F)F)cc1)c1ccccc1 ; CCC(=O)N(C)CCC(Oc1ccc(C(F)(F)F)cc1)c1ccccc1 ; CN(CCC(Oc1ccc(C(F)(F)F)cc1)c1ccccc1)S(C)(=O)=O ; Cc1ccc(S(=O)(=O)N(C)CCC(Oc2ccc(C(F)(F)F)cc2)c2ccccc2)cc1]

or a pharmaceutically acceptable salt thereof.

In some embodiments, the lung disease is chronic obstructive pulmonary disease.

In some embodiments, the compound is administered in a pharmaceutical composition comprising the compound and a pharmaceutically acceptable carrier.

In some embodiments, the compound is delivered orally.

In some embodiments, the compound is delivered by direct delivery to the lung. In some embodiments, the direct delivery is by inhalation.

In some embodiments, the compound inhibits cigarette smoke induced MMP-1 production in small airway epithelial cells. In some embodiments, the inhibition is increased by at least 50% as compared with inhibition in a subject that has been administered fluoxetine. In some embodiments, the inhibition is increased by at least 75% as compared with inhibition in a subject that has been administered fluoxetine. In some embodiments, the inhibition is increased by at least 90% as compared with inhibition in a subject that has been administered fluoxetine.

In some embodiments, the treating comprises prophylactic treatment.

The present invention also provides an inhaler containing a compound or pharmaceutical composition as described herein.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### Terms

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

As used herein, “about” in the context of a numerical value or range means.+−0.10% of the numerical value or range recited or claimed, unless the context requires a more limited range.

As used herein, the term “composition”, as in pharmaceutical composition, is intended to encompass a product comprising the active ingredient(s) and the inert ingredient(s) that make up the carrier, as well as any product which results, directly or indirectly from combination, complexation, or aggregation of any two or more of the ingredients, or from dissociation of one or more of the ingredients, or from other types of reactions or interactions of one or more of the ingredients.

As used herein, “effective amount” refers to an amount which is capable of treating a subject having a disease or a disorder. Accordingly, the effective amount will vary with the subject being treated, as well as the condition to be treated. A person of ordinary skill in the art can perform routine titration experiments to determine such sufficient amount. The effective amount of a compound will vary depending on the subject and upon the particular route of administration used. Based upon the compound, the amount can be delivered continuously, such as by continuous pump, or at periodic intervals (for example, on one or more separate occasions). Desired time intervals of multiple amounts of a particular compound can be determined without undue experimentation by one skilled in the art. In one embodiment, the effective amount is between about 1 μg/kg-10 mg/kg. In another embodiment, the effective amount is between about 10 μg/kg-1 mg/kg. In a further embodiment, the effective amount is 100 μg/kg.

“Subject” shall mean any organism including, without limitation, a mammal such as a mouse, a rat, a dog, a guinea pig, a ferret, a rabbit and a primate. In one embodiment, the subject is a human.

“Treating” means either slowing, stopping or reversing the progression of a disease or disorder. As used herein, “treating” also means the amelioration of symptoms associated with the disease or disorder. Treating can include prophylactic treatment.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, “approved for use in human subjects” means approved for any medicinal use in human subjects at any time by any government agency of any country. In some embodiments, a compound that has been approved for use in human subjects was approved by the Food and Drug Administration (FDA) of the United States. For example, an SSRI that is approved for use in human subjects may in some embodiments be an SSRI that is approved for use in treating depression by the FDA.

Non-limiting examples of commercially available SSRIs include Duloxetine and Fluoxetine.

Duloxetine is an SSRI that is commercially available from Eli Lilly and Company (Indianapolis, Ind. 46285). The ChemSpider identification number for Duloxetine is 54822. Duloxetine is also known as Cymbalta. Duloxetine is described in Perahia et al. (2006) Duloxetine 60 mg once daily in the treatment of milder major depressive disorder. Int. J. Clin. Pract. 60 (5): 613-20 and U.S. Pat. No. 8,269,023, the entire contents of each of which are hereby incorporated herein in their entireties.

Fluoxetine is an SSRI that is commercially available from Eli Lilly and Company (Indianapolis, Ind. 46285). The CAS Registry number for Fluoxetine is 54910-89-3. Fluoxetine is also known as Lilly-110140, Sarafem, and Prozac (fluoxetine hydrochloride). Fluoxetine is described in Altamura et al. (1994). Clinical Pharmacokinetics of Fluoxetine. Clinical Pharmacokinetics 26 (3): 201-214 and U.S. Pat. No. 5,166,437, issued Nov. 24, 1992, the entire contents of each of which are hereby incorporated herein in their entireties.

Aspects of the present invention relate to compounds that inhibit the induction of MMP-1 expression by cigarette smoke or cigarette smoke extract (CSE). In some embodiments, compounds that block more than 80% and no more than 120% of cigarette or CSE induced MMP-1 expression are selected for use in treating subjects (inhibition greater than 120% would indicate baseline inhibition of MMP-1 expression unrelated to CSE). Therefore, aspects of the present invention relate to SSRIs that reduce the induction of MMP-1 by cigarette smoke or CSE without reducing baseline MMP-1 expression more than 5, 10, 15, or 20%.

In some embodiments, and depending on the assay used, the percentage inhibition of the CSE/MMP-1 induction may be calculated for compounds on a per-plate basis, using the equation: % inhibition of compound=100×[1−(test well-median high-signal control)/(median high-signal control-median low-signal control)].

It will be understood by persons skilled in the art that the percent inhibition of MMP-1 induced expression may be assayed using the methods described in the Examples herein. It will also be understood that assays other than the methods exemplified herein, or variations thereof, may be used to determine the percent inhibition of the induced expression of MMP-1. Non-limiting examples of other methods for assaying the induced expression of MMP-1 (and the inhibition thereof) include quantitative real-time PCR (qPCR), Western Blot analysis, Northern Blot, and array analysis (such as microarray analysis).

Ester derivatives of compounds used in the subject invention may be generated from a carboxylic acid group in accordance with the present invention using standard esterification reactions and methods readily available and known to those having ordinary skill in the art of chemical synthesis. Ester derivatives may serve as pro-drugs that can be converted into compounds by serum esterases.

Compounds used in the methods of the present invention may be prepared by techniques well known in organic synthesis and familiar to a practitioner ordinarily skilled in the art. However, these may not be the only means by which to synthesize or obtain the desired compounds.

Compounds used in the methods of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5th Edition (2007), and references therein, which are incorporated by reference herein. However, these may not be the only means by which to synthesize or obtain the desired compounds.

In some embodiments, a compound may be in a salt form. As used herein, a “salt” is a salt of the instant compound which has been modified by making acid or base salts of the compounds. In the case of the use of compounds for treatment of a disease, the salt is pharmaceutically acceptable. Examples of pharmaceutically acceptable salts include, but are not limited to, mineral or organic acid salts of basic residues such as amines. The term “pharmaceutically acceptable salt” in this respect, refers to the relatively non-toxic, inorganic and organic base addition salts of compounds. These salts can be prepared in situ during the final isolation and purification of a compound, or by separately reacting a purified compound in its free acid form with a suitable organic or inorganic base, and isolating the salt thus formed.

The compounds used in some embodiments of the present invention can be administered in a pharmaceutically acceptable carrier. As used herein, a “pharmaceutically acceptable carrier” is a pharmaceutically acceptable solvent, suspending agent or vehicle, for delivering the compounds to the subject. The carrier may be liquid or solid and is selected with the planned manner of administration in mind. Liposomes are also a pharmaceutically acceptable carrier. The compounds used in the methods of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form.

Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

“Administering” compounds in embodiments of the invention can be effected or performed using any of the various methods and delivery systems known to those skilled in the art. The administering can be, for example, intranasal, intravenous, oral, intramuscular, intravascular, intra-arterial, intracoronary, intramyocardial, intraperitoneal, and subcutaneous. Aspects of the present invention relate to the nasal or oral inhalation of a compound using an inhaler. Other non-limiting examples include topical administration, or coating of a device to be placed within the subject. In some embodiments, administration is effected by injection or via a catheter.

Aspects of the present invention relate to the administration of a compound using an inhaler. In some embodiments, an amount of a compound-containing aerosol or powder is discharged into the nose or mouth of a subject using an inhaler. Non-limiting examples of inhalers are described in U.S. Pat. No. 7,900,625, issued Mar. 8, 2011; U.S. Pat. No. 5,891,419, issued Apr. 6, 1999; U.S. Pat. No. 3,456,644, issued Jul. 22, 1969; U.S. Pat. No. 6,684,879, issued Feb. 3, 2004; U.S. Pat. No. 7,448,385, issued Nov. 11, 2008; U.S. Pat. No. 8,555,878, issued Oct. 15, 2013; U.S. Pat. No. 7,073,499, issued Jul. 11, 2006; and PCT International Patent Application Publication No. 2014/137215, published Sep. 12, 2014.

Inhaled delivery of pulmonary drugs offers clear advantages over oral or systemic preparations. Importantly, inhibitors against other MMPs as administered by traditional routes have stalled in clinical trials due to untoward systemic toxicity.

Injectable drug delivery systems may be employed in the methods described herein include solutions, suspensions, and gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

The dosage of a compound administered in treatment will vary depending upon factors such as the pharmacodynamic characteristics of the compound and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of a compound may comprise a compound alone, or mixtures of a compound with additional compounds used to treat a disease, e.g. COPD. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or inhalation or other methods, into the lung, all using dosage forms well known to those of ordinary skill in the pharmaceutical arts.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

A compound may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, a compound may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain a compound and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, a compound may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

A compound may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

Where a range is given in the specification it is understood that the range includes all integers and 0.1 units within that range, and any sub-range thereof. For example, a range of 1 to 5 is a disclosure of 1.0, 1.1, 1.2, etc.

The present invention is advantageous over the art in that it provides novel compounds directly targeting the pathogenic processes responsible for lung destruction in COPD, and not simply treating the symptoms of disease. We have identified compounds that block two important smoke induced pathways, protease production and inflammation. The use of such compounds is exclusive in the field of COPD with the ability to actually block pathways important in actively degrading and damaging the lung secondary to cigarette smoke or other inflammatory injuries. These compounds will therefore benefit not only severely affected COPD patients but potentially target all patients with COPD to stabilize disease and protect the lung from further destruction.

Our preliminary data has led us to target selective serotonin reuptake inhibitors (SSRI) as potential therapeutic agents for COPD. Already FDA approved for depression, oral SSRI administration has a long and proven track record of safety. Despite this, chronic SSRI treatment is associated with many side effects, such as weight gain and sexual dysfunction, that limit treatment compliance. To limit side effects via decreased central nervous system penetration and dose reduction while increasing efficacy, we develop novel inhaled SSRI derivatives for direct delivery to the lung as a disease modifying treatment for COPD.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

The compounds described herein can be prepared using standard techniques of synthetic organic chemistry, well known to those of ordinary skill in the art. Derivative compounds of fluoxetine were prepared via demethylation and substitution at the terminal nitrogen with various functional groups.

### Example 1

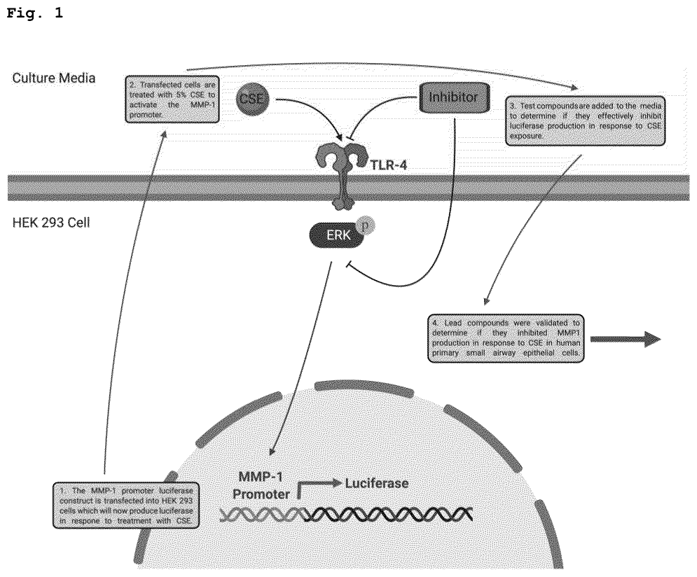

Preliminary Studies: Our aim is directed to developing molecules that can modulate the transcriptional induction of lung damaging MMPs induced by cigarette smoke. Prior work from our academic collaborator utilized a mammalian cell-based assay based on transfection of a human cell line (HEK 293T) with a vector containing a luciferase reporter gene under the control of the MMP1 promoter (FIG. 1). This method is based on the fact that the MMP1 promoter contains a specific cigarette smoke responsive element (CRE). Utilizing the MMP1 promoter, an MMP1/pGL3 luciferase reporter vector was prepared and transfected into cells. The assay was developed by treating transfected cells with cigarette smoke extract (CSE), tested for reproducibility and inhibition with MAPKinase inhibitors that were known to block smoke induced MMP1 expression. Utilizing this assay, we tested the effect of a collection of 727 structurally diverse small molecules obtained from the NIH clinical collection. A suitable assay is further described in U.S. Patent Application Publication No. US 2018/0028493, incorporated by reference herein in its entirety.

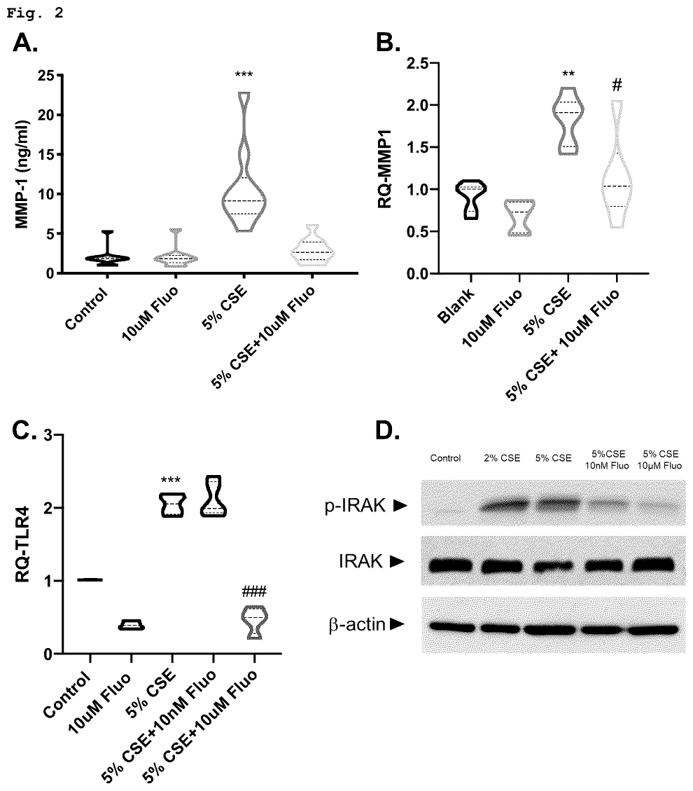

The compounds in this clinical set have all been tested and utilized in humans for various indications. These molecules were tested for their capacity to modulate MMP-1 smoke induced transcriptional activation. It was determined that the SSRIs duloxetine and fluoxetine were potent inhibitors of cigarette smoke induced MMP-1 production. Prior work in our lab was critical in establishing MMP-1 as a destructive agent in the progression of COPD in humans. As such, we next exposed human small airway epithelial cells to cigarette smoke extract followed by treatment with fluoxetine. Fluoxetine treated cultures were characterized by decreased MMP-1 expression and secretion in addition to decreased expression of the inflammatory mediators TLR-4 and phospho-IRAK, its downstream effector (FIG. 2). Together, these data support our hypothesis that SSRIs inhibit the epithelial protease and inflammatory response to cigarette smoke.

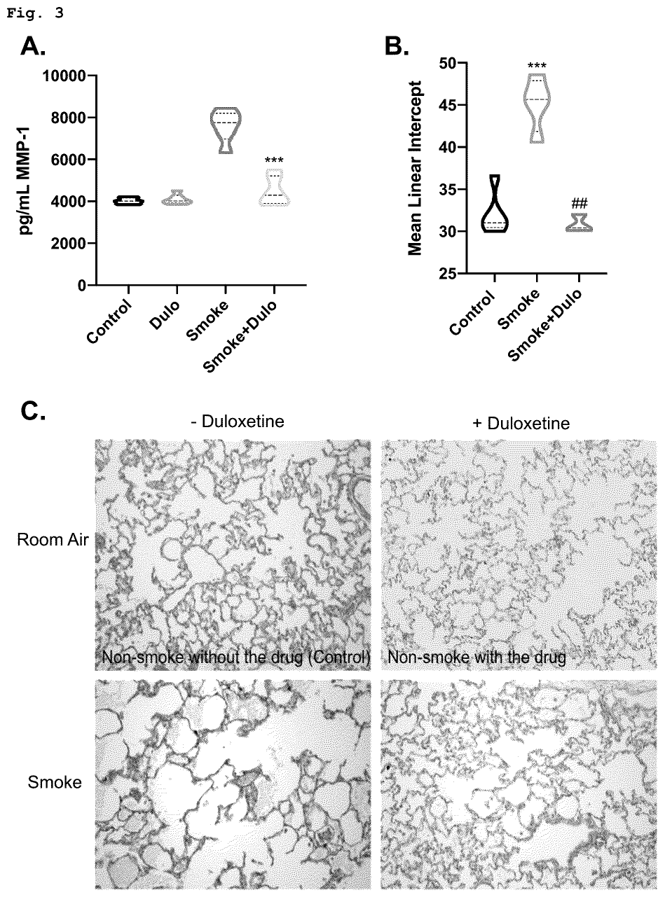

As the next step, we observed the ability of oral duloxetine to inhibit cigarette smoke induced up-regulation of MMP1 and lung destruction in a rabbit model. Rabbits were utilized for these experiments since unlike mice they produce MMP-1. Rabbits were exposed to cigarette smoke for 16 weeks while receiving placebo or orally administered duloxetine. At 16 weeks, bronchoalveolar lavage fluid was assessed for MMP1 activity by ELISA and as shown in FIG. 3A drug treatment reduced MMP1 levels.

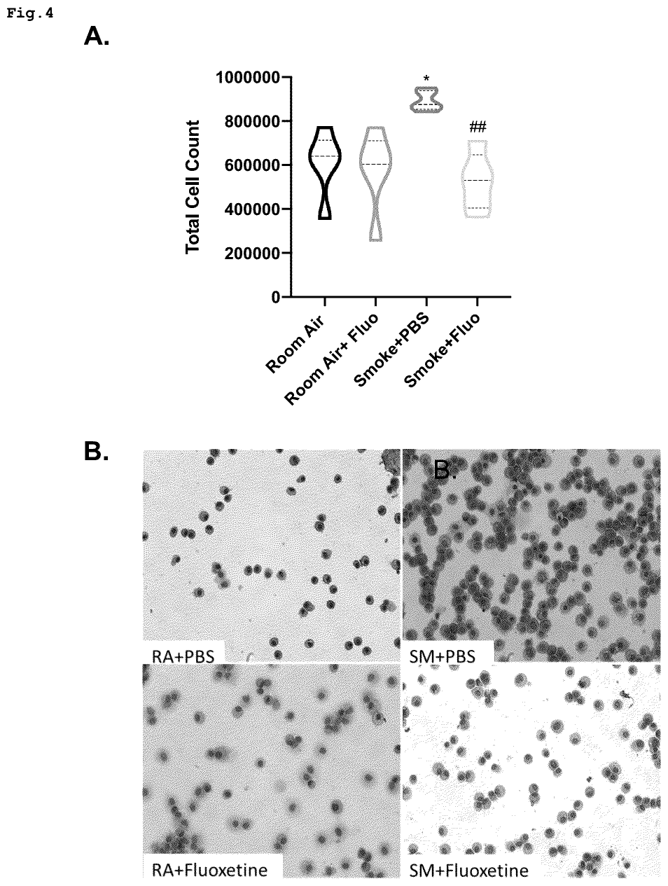

Histological analysis revealed decreased lung destruction in rabbits receiving duloxetine, suggesting inhibition of MMP1 expression prevents protease induced lung injury (FIG. 3C). This observation was confirmed by morphometric analysis which showed decreased mean linear intercepts for animals receiving duloxetine plus smoke as opposed to smoke alone (FIG. 3B). Taken together, these data demonstrate that, SSRI induced suppression of MMP1 expression in response to cigarette smoke prevents MMP1 mediated lung destruction and emphysema development, supporting their further development as inhaled agents for COPD treatment. To assess the effects of SSRIs on the cigarette smoke induced inflammatory response, we utilized smoke-exposed mice. Although mice do not express MMP1, they exhibit a robust inflammatory response to cigarette smoke and activated the signaling pathways that drive MMP-1 expression in rabbits and humans. Consistent with our above findings, mice exposed to cigarette smoke for 10 days and treated with fluoxetine exhibited decreased inflammatory cell counts in lung lavage fluid. (FIG. 4).

### Example 2

Aim 1. This aim begins the process of chemical lead (CL) identification through examination of the structure-activity relationship (SAR) of fluoxetine and confirm the effectiveness of potential candidates using secondary mechanistic assays.

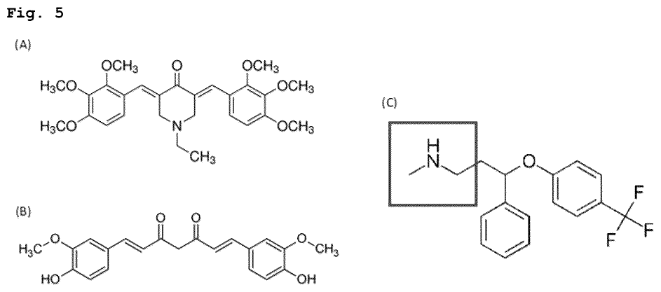

Aim 1a: Determining if systematic chemical modification of fluoxetine produces a CL hit (s) with an enhanced ability to inhibit MMP1 transcriptional activation. An existing library of fluoxetine derived compounds (KayBare Bio, NJ) is screened to examine the SAR of the parent molecule. We hypothesize that fluoxetine binds to the MD2 pocket of the TLR4 receptor based on structural similarity to the small molecule inhibitors curcumin and L48H37 (FIG. 5). Given this, the first derivatives examined consist of 6 molecules with alterations made to the terminal secondary amine group of fluoxetine (Table 1).

A high throughput screen (HTS), validated and developed in our laboratory, is used to examine the ability of these compounds to inhibit MMP1 transcription. Briefly, HEK293 cells are transformed with the cigarette smoke responsive MMP1-promoter/pGL3 luciferase plasmid, cultured in 96-well plates and exposed to CSE to activate the reporter construct. Each derivative and fluoxetine, dissolved in DMSO, is tested at a concentration of 10 μM in an interleaved format to assess its ability to antagonize CSE-induced MMP-1 transcription using the CellTiter-Glo (Promega Corp) assay. Using the following equation, % inhibition of compound=100×[1−(test well−median high-signal control)/(median high-signal control−median low-signal control)], inhibition is calculated on a per-plate basis for each compound examined. A result is considered a positive hit if a molecule shows at least a 50% increase in inhibition as compared to fluoxetine. For each potential lead-compound, a 10-point series of 1:3 dilutions is tested in triplicate to construct a dose-response curve (DRC) for IC₅₀ determination and Hill slope determination.

TABLE 1
SSRI Derivative Compounds

| | |
|---|---|
| 1. | 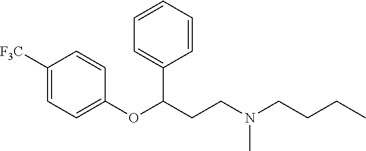 [SMILES: CCCCN(C)CCC(Oc1ccc(C(F)(F)F)cc1)c1ccccc1] |
| | N-methyl-N-(3-phenyl-3-(4- |
| | (trifluoromethyl)phenoxy)propyl)butan-1-amine |
| 2. | 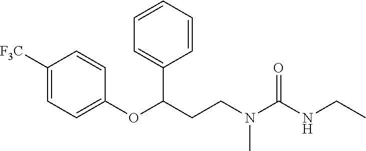 [SMILES: CCNC(=O)N(C)CCC(Oc1ccc(C(F)(F)F)cc1)c1ccccc1] |
| | 3-ethyl-1-methyl-1-(3-phenyl-3-(4- |
| | (trifluoromethyl)phenoxy)propyl)urea |
| 3. | 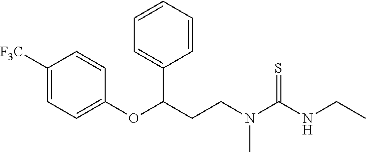 [SMILES: CCNC(=S)N(C)CCC(Oc1ccc(C(F)(F)F)cc1)c1ccccc1] |
| | 3-ethyl-1-methyl-1-(3-phenyl-3-(4- |
| | (trifluoromethyl)phenoxy)propyl)thiourea |
| 4. | 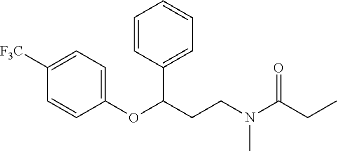 [SMILES: CCC(=O)N(C)CCC(Oc1ccc(C(F)(F)F)cc1)c1ccccc1] |
| | N-methyl-N-(3-phenyl-3-(4- |
| | (trifluoromethyl)phenoxy)propyl)propionamide |
| 5. | 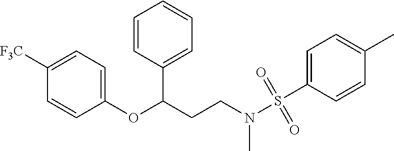 [SMILES: Cc1ccc(S(=O)(=O)N(C)CCC(Oc2ccc(C(F)(F)F)cc2)c2ccccc2)cc1] |
| | N,4-dimethyl-N-(3-phenyl-3-(4- |
| | (trifluoromethyl)phenoxy)propyl)benzenesulfonamide |
| 6. | 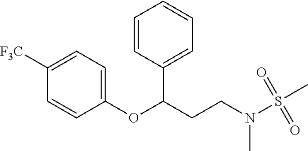 [SMILES: CN(CCC(Oc1ccc(C(F)(F)F)cc1)c1ccccc1)S(C)(=O)=O] |
| | N-methyl-N-(3-phenyl-3-(4- |
| | (trifluoromethyl)phenoxy)propyl)methanesulfonamide |

Aim 1b: Determining if potential CL(s) maintain their ability to inactivate MMP1 transcription and inflammatory pathways via inhibition of TLR4 signaling in human small airway epithelial cells. For each positive hit and fluoxetine, the DRC is used to derive their respective IC₅₀'s and Hill slopes. The concentration required to achieve a 50%, 75% and 99.9% fractional effect is calculated using the formula: [C_{f}]=[(fraction/100-fraction) ^{(1/Hill slope)}]IC₅₀. The effect of each calculated concentration on cell viability is tested in triplicate on primary human small airway epithelial cell (hSAEC) culture via MTT assay. Dosing and fractional effect are adjusted accordingly to ensure SAEC viability during experimentation based on these results. The chosen concentrations are tested on SAEC cultures exposed to 5% cigarette smoke extract for 24 h to assess their effects on MMP1 and TLR4 expression via mRNA extraction to analyze transcript levels by real-time RT-PCR. Culture media is collected and MMP1 secretion assessed by ELISA (R&D Systems, MN). Protein collected for western blot is used to assess IRAK phosphorylation, a known downstream effector of TLR4 activity. In addition, PCR arrays (ThermoFisher, MA) that measure the expression of cytokines that have been implicated in the pathogenesis of COPD, their receptors and the expression of transcription factors that regulate the cellular response to oxidants and CSE exposure are used to determine the ability of each derivative to suppress inflammatory signaling in smoke exposed cells.

Statistical analysis and Power: All experiments are assayed in triplicates, and each experiment is repeated at least three times. (Statistical analysis of the data obtained in this part of the proposal is performed using the unpaired two-tailed Student's T-test if 2 groups are being compared). In the case of a comparison between three or more groups one-way ANOVA followed by the Bonferroni post hoc test is performed using GraphPad Prism software.

Aim 2: Determining if CL hits and fluoxetine cause unacceptable pulmonary toxicity in vitro. Any new hits uncovered in aim 1 have unknown toxicity profiles in the lung and other organ systems. In addition, although widely utilized systemically with an excellent safety profile, effects of fluoxetine's direct application to the pulmonary epithelium have not been explored. Non-GLP in vitro toxicity testing utilizing pulmonary air liquid interface (ALI) cultures is employed to eliminate potential CLs that have a risk of adverse effects. ALI cultures replicate the ciliated pseudostratified pulmonary epithelium and its barrier properties with high fidelity in vitro. EpiAirway (Matek Life Sciences, MA) ALI cultures are purchased ready to use. A 1:3 10-point dilution series is created for dose response curve construction, with cultures exposed to the appropriate concentration for 24 h. Cytotoxicity is assessed by multiplexing stains for mitochondrial function and apoptosis; MitoTracker red CMXRos (Thermofisher, MA) and Cell Event caspase 3/7 activity assay (Thermofisher, MA) is used to detect changes in mitochondrial function and epithelial cell apoptosis, respectively.

Signal is detected for both reagents by microplate reader and imaging via fluorescent confocal microscopy. In addition, culture media is collected from the apical and basal chambers of each culture and degree of lactate dehydrogenase (LDH) release is determined using the CyQUANT LDH Cytotoxicity Assay (Thermofisher, MA). Combined, these staining techniques should provide sufficient information to determine the potential degree of toxicity to the lung allowing us to eliminate harmful compounds early in the development process.

Statistical analysis and Power: All experiments are assayed in triplicates, and each experiment is repeated at least three times. (Statistical analysis of the data obtained in this part of the proposal is performed using the unpaired two-tailed Student's T-test if 2 groups are being compared. In the case of a comparison between three or more groups one-way ANOVA followed by the Bonferroni post hoc test is performed using GraphPad Prism software.

Apart from its role in emphysema formation described above, MMP1 has been implicated in several pathological processes, including tumor invasion, arthritis, skin repair and atherosclerotic plaque rupture. Therefore, the small molecules identified in this study have wide applicability for the treatment of various diseases.

### Example 3

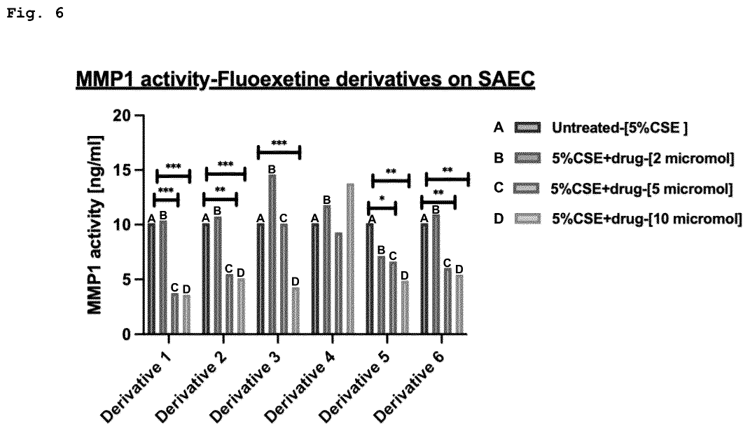

As shown in FIG. 6, derivatives 1,2,5 and 6 significantly decreasd MMP1 concentrations at higher concentrations:
Derivative 1 5 μM−*** p value=0.0006, 10 μM−*** p value=0.001
Derivative 2 5 μM−** p value=0.007, 10 μM−*** p value=0.0018
Derivative 5 5 μM−* p value=0.02, 10 μM−** p value=0.003
Derivative 6 5 μM−** p value=0.003, 10 μM−** p value=0.005.

Derivative 3 did not show any reduction of MMP1 in both 2 and 5 μM concentrations, but 10 μM showed significant reduction of MMP1; ***p value=0.0005. Derivative 4 did not show any reduction of MMP1 at any of the three concentrations. No significant reduction of MMP1 was seen for any derivative at 2 μM concentration.

### Example 4

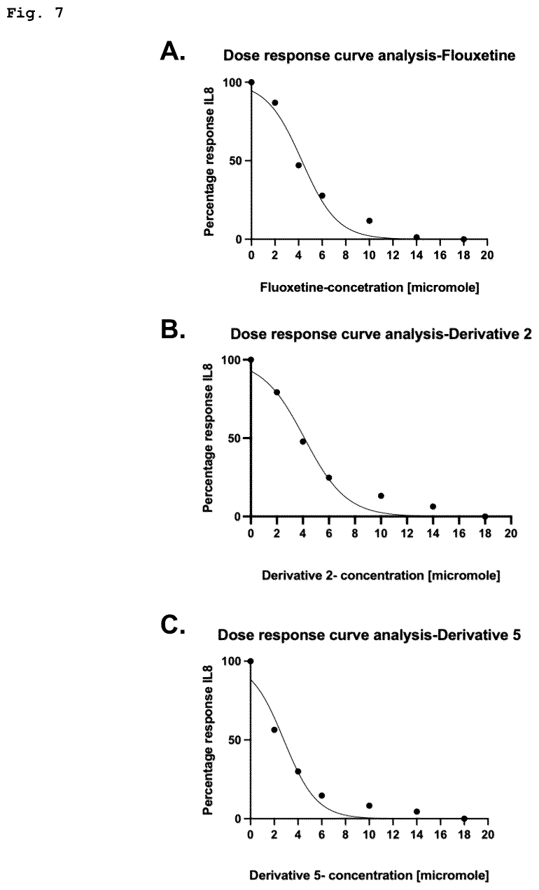

As shown in FIG. 7, Fluoxetine derivative treatment reduces IL-8 expression in CSE treated SAEC. SAEC cultures were treated with 5% CSE and varying doses of Fluoxetine, Derivative 2 and Derivative 5, respectively. Treatment with the derivatives decreased MMP1 induction after cigarette smoke treatment.

### Example 5

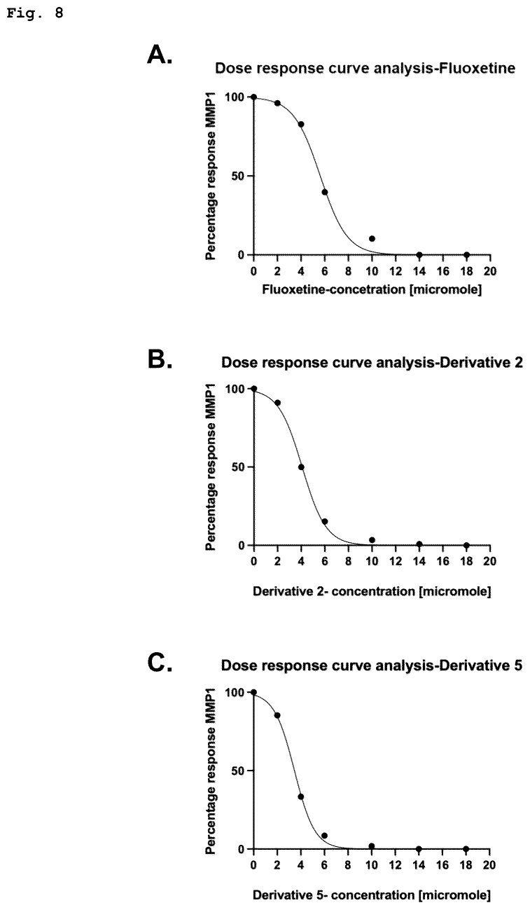

As shown in FIG. 8, Fluoxetine derivative treatment reduces MMP1 expression in CSE treated SAEC. SAEC cultures were treated with 5% CSE and varying doses of Fluoxetine, Derivative 2 and Derivative 5, respectively. Treatment with the derivatives decreased MMP1 induction after cigarette smoke treatment.

What is claimed is:

1. A compound having the structure: 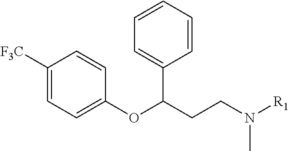
wherein
R₁ is —C(O)—NH-(alkyl), —SO₂-(alkyl)
wherein the alkyl may be straight, branched or cyclic
or —SO₂-(aryl)
wherein the aryl is optionally substituted with one or more -(alkyl), —O-(alkyl), halogen, —CF₃, —OCF₃ or —CN
or a pharmaceutically acceptable salt thereof.

2. The compound of claim 1 having the structure: 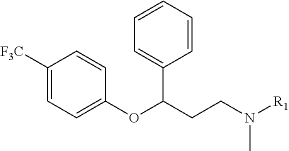
wherein
R₁ is —C(O)—NH-(alkyl) or —SO₂-(alkyl),
wherein the alkyl may be straight, branched or cyclic;
or a pharmaceutically acceptable salt thereof.

3. The compound of claim 2, wherein R₁ is —C(O)—NH—(C₁-C₈ alkyl) or —SO₂— (C₁-C₈ alkyl)

4. A compound having the structure: 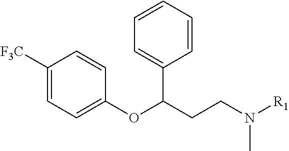
wherein
R₁ is —C(O)—NH-(ethyl) or —SO₂-(methyl);
or a pharmaceutically acceptable salt thereof.

5. The compound of any one of claims 2-4, wherein
R₁ is —C(O)—NH-(ethyl).

6. The compound of any one of claims 2-4, wherein
R₁ is —SO₂-(methyl).

7. The compound of claim 1, wherein
R₁ is —SO₂-(4-methylphenyl).

8. The compound of any one of claims 2-6 having the structure: 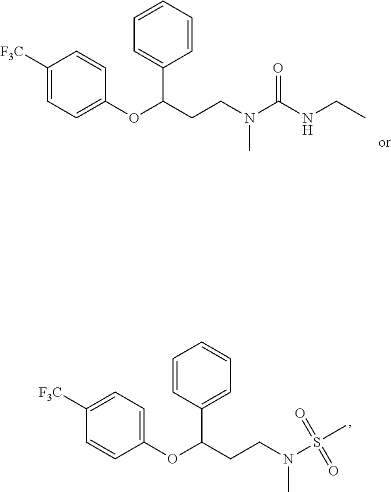 [SMILES: CCNC(=O)N(C)CCC(Oc1ccc(C(F)(F)F)cc1)c1ccccc1 ; CN(CCC(Oc1ccc(C(F)(F)F)cc1)c1ccccc1)S(C)(=O)=O]
or a pharmaceutically acceptable salt thereof.

9. The compound of any one of claims 1 or 7 having the structure: 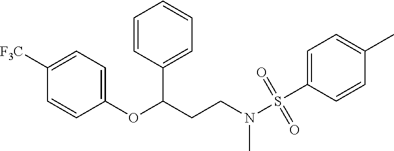 [SMILES: Cc1ccc(S(=O)(=O)N(C)CCC(Oc2ccc(C(F)(F)F)cc2)c2ccccc2)cc1]
or a pharmaceutically acceptable salt thereof.

10. A pharmaceutical composition comprising the compound of any one of claims 2-6 or 8 and a pharmaceutically acceptable carrier.

11. A pharmaceutical composition comprising the compound of any one of claims 1, 7 or 9 and a pharmaceutically acceptable carrier.

12. A method of treating a subject afflicted with a lung disease comprising administering to the subject an effective amount of a compound having the structure: 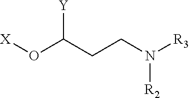
wherein
X is aryl or heteroaryl, which is optionally substituted with one or more -(alkyl), —O-(alkyl), halogen, —CF₃, —OCF₃ or —CN, wherein the alkyl may be straight, branched or cyclic;
Y is aryl or heteroaryl, which is optionally substituted with one or more -(alkyl), —O-(alkyl), halogen, —CF₃, —OCF₃ or —CN, wherein the alkyl may be straight, branched or cyclic;
R₂ is H, -(alkyl), -(alkenyl), -(alkynyl) or -cycloalkyl, wherein the alkyl, alkenyl or alkynyl may be straight, branched or cyclic; and
R₃ is H, -(alkyl), -(alkenyl), -(alkynyl), —C(O)—NH-(alkyl), —C(S)—NH-(alkyl), —C(O)-(alkyl), —SO₂-(alkyl) or —SO₂-(aryl), wherein the alkyl, alkenyl or alkynyl may be straight, branched or cyclic, and the aryl is optionally substituted with one or more -(alkyl), —O-(alkyl), halogen, —CF₃, —OCF₃ or —CN;
or a pharmaceutically acceptable salt thereof, so as to thereby treat the subject.

13. The method of claim 12, wherein
X is aryl, optionally substituted with one or more -(alkyl), —O-(alkyl), halogen, —CF₃, —OCF₃ or —CN, wherein the alkyl may be straight, branched or cyclic.

14. The method of claim 12 or 13, wherein Y is aryl, optionally substituted with one or more -(alkyl), —O-(alkyl), halogen, —CF₃, —OCF₃ or —CN, wherein the alkyl may be straight, branched or cyclic.

15. The method of any one of claims 12-14, wherein
X is phenyl, or
Y is phenyl, or
X and Y are phenyl.

16. The method of any one of claims 12-15, wherein
R₂ is -(alkyl).

17. The method of any one of claims 12-16, wherein
R₃ is -(alkyl), —C(O)—NH-(alkyl), —C(S)—NH-(alkyl), —C(O)-(alkyl), —SO₂-(alkyl) or —SO₂-(aryl).

18. The method of any one of claims 12-17, wherein the compound has the structure: 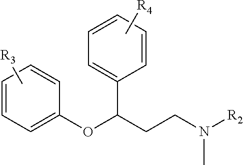
wherein
R₂ is -(alkyl), —C(O)—NH-(alkyl), —C(S)—NH-(alkyl), —C(O)-(alkyl), —SO₂-(alkyl) or —SO₂-(aryl); and
R₃ and R₄ are each independently H, -(alkyl), —O-(alkyl), halogen, —CF₃, —OCF₃ or —CN, wherein the alkyl may be straight, branched or cyclic,
or a pharmaceutically acceptable salt thereof.

19. The method of any one of claims 12-18, wherein the compound has the structure: 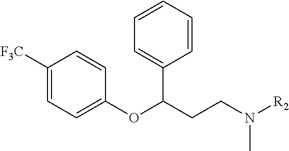
wherein
R₂ is -(alkyl), —C(O)—NH-(alkyl), —C(S)—NH-(alkyl), —C(O)-(alkyl), —SO₂-(alkyl) or —SO₂-(aryl),
or a pharmaceutically acceptable salt thereof.

20. The method of any one of claims 12-19, wherein:
R₂ is -(n-butyl), —C(O)—NH-(ethyl), —C(S)—NH-(ethyl), —C(O)-(ethyl), —SO₂-(methyl) or —SO₂-(4-methylphenyl).

21. The method of any one of claims 12-20, wherein the compound has the structure: 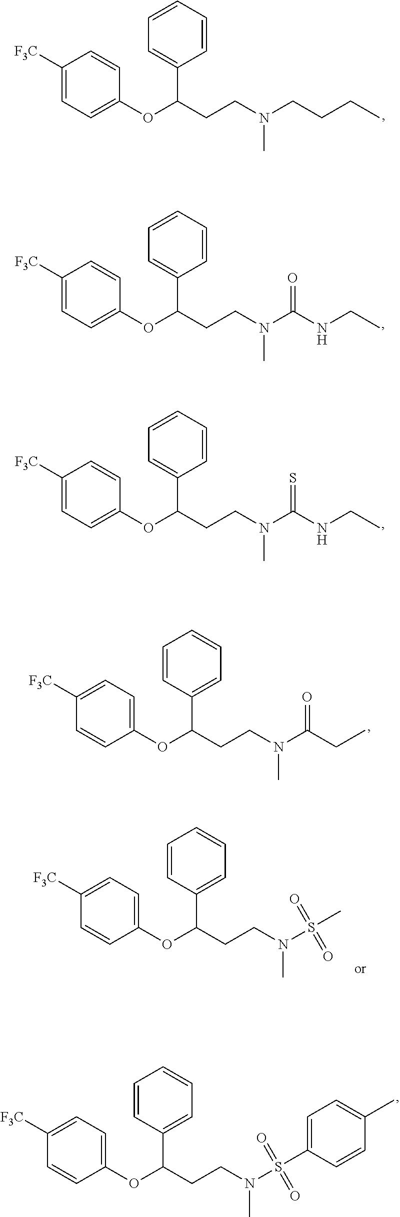 [SMILES: CCCCN(C)CCC(Oc1ccc(C(F)(F)F)cc1)c1ccccc1 ; CCNC(=O)N(C)CCC(Oc1ccc(C(F)(F)F)cc1)c1ccccc1 ; CCNC(=S)N(C)CCC(Oc1ccc(C(F)(F)F)cc1)c1ccccc1 ; CCC(=O)N(C)CCC(Oc1ccc(C(F)(F)F)cc1)c1ccccc1 ; CN(CCC(Oc1ccc(C(F)(F)F)cc1)c1ccccc1)S(C)(=O)=O ; Cc1ccc(S(=O)(=O)N(C)CCC(Oc2ccc(C(F)(F)F)cc2)c2ccccc2)cc1]
or a pharmaceutically acceptable salt thereof.

22. The method of any one of claims 12-21, wherein the lung disease is chronic obstructive pulmonary disease.

23. The method of any of claims 12-22, wherein the compound is administered in a pharmaceutical composition comprising the compound and a pharmaceutically acceptable carrier.

24. The method of any of claims 12-23, wherein the compound is delivered orally.

25. The method of any of claims 12-23, wherein the compound is delivered by direct delivery to the lung.

26. The method of claim 25, wherein the direct delivery is by inhalation.

27. The method of any of claims 12-26, wherein the compound inhibits cigarette smoke induced MMP-1 production in small airway epithelial cells.

28. The method of claim 27, wherein the inhibition is increased by at least 50% as compared with inhibition in a subject that has been administered fluoxetine.

29. The method of claim 27, wherein the inhibition is increased by at least 75% as compared with inhibition in a subject that has been administered fluoxetine.

30. The method of claim 27, wherein the inhibition is increased by at least 90% as compared with inhibition in a subject that has been administered fluoxetine.

31. The method of any of claims 12-30, wherein the treating comprises prophylactic treatment.

32. An inhaler containing a compound of any of claims 2-6 or 8 or the pharmaceutical composition of claim 8.

33. An inhaler containing a compound of any of claims 1, 7 or 9 or the pharmaceutical composition of claim 9.
